(19) United States
(12) Patent Application Publication
(10) Pub. No.: US 2025/0228550 A1
(43) Pub. Date: Jul. 17, 2025

(54) LOCKING FERRULES FOR PERFORMING TENSIONABLE KNOTLESS SURGICAL PROCEDURES

(71) Applicant: ARTHREX, INC., Naples, FL (US)

(72) Inventors: Peter J. Dreyfuss, Naples, FL (US); Joshua Dines, New York, NY (US); Gautam Yagnik, Miami, FL (US)

(21) Appl. No.: 18/419,862

(22) Filed: Jan. 23, 2024

Related U.S. Application Data
(60) Provisional application No. 63/619,837, filed on Jan. 11, 2024.

(51) Int. Cl. A61B 17/04

(52) U.S. Cl. CPC A61B 17/0487

(57) ABSTRACT

Systems and methods are provided for performing tensionable knotless tissue repairs. A locking ferrule that includes a one-way locking mechanism may be utilized as part of the tensionable knotless tissue repairs for tensioning and locking one or more strands of suture. The one-way locking mechanism may be established by one or more locking barbs of the locking ferrule. The locking ferrule may further include an eyelet or a through-bore for accommodating the suture.

### BACKGROUND

This application claims the benefit of U.S. Provisional Application No. 63/619,837, which was filed on Jan. 11, 2024 and is incorporated herein by reference in its entirety.

### BACKGROUND

This disclosure relates to the field of surgery, and more particularly to a locking ferrule that includes an integrated eyelet or transverse through-bore for accommodating suture. The locking ferrule is configured for locking the suture when performing a variety of tensionable knotless tissue repairs.

Repetitive trauma to a joint, such as a knee, ankle, hip, or shoulder joint, for example, may result in the development of tissue defects (e.g., soft tissue tears, cartilage defects, etc.). If not treated, tissue defects could further deteriorate, thereby causing joint instability and discomfort.

### SUMMARY

This disclosure relates to systems and methods for performing tensionable knotless tissue repairs. A locking ferrule that includes a one-way locking mechanism may be utilized as part of the tensionable knotless tissue repairs for tensioning and locking one or more strands of suture.

An exemplary locking ferrule for performing tissue repairs may include, inter alia, a body extending between a first end and a second end and including an outer diameter wall, an inner diameter wall, and a cannulation that is circumscribed by the inner diameter wall. An eyelet is connected to the body. A plurality of locking barbs extend into the cannulation and are configured to lock a suture that is received through the eyelet and the cannulation relative to the body.

An exemplary surgical method may include, inter alia, loading a suture through an eyelet of a locking ferrule, passing the suture through a cannulation of the locking ferrule, tensioning the suture in a first direction, and locking the suture within the cannulation to prevent movement of the suture in a second direction.

Another exemplary locking ferrule for performing tissue repairs may include, inter alia, a body including an outer diameter wall, an inner diameter wall, and a cannulation that is circumscribed by the inner diameter wall. A plurality of locking barbs extend into the cannulation and are configured to lock a suture that is received through the cannulation relative to the body. A transverse through-bore extends through the body between a first row and a second row of the plurality of locking barbs.

The embodiments, examples, and alternatives of the preceding paragraphs, the claims, or the following description and drawings, including any of their various aspects or respective individual features, may be taken independently or in any combination. Features described in connection with one embodiment are applicable to all embodiments, unless such features are incompatible.

The various features and advantages of this disclosure will become apparent to those skilled in the art from the following detailed description. The drawings that accompany the detailed description can be briefly described as follows.

### BRIEF DESCRIPTION OF THE DRAWINGS

FIG. 1 illustrates a locking ferrule that can be used for performing various tensionable knotless tissue repairs.

FIG. 2 is a cross-sectional view through section 2-2 of FIG. 1.

FIG. 3 is a cross-sectional view through section 3-3 of FIG. 2.

FIG. 4 schematically illustrates locking a suture relative to the locking ferrule of FIGS. 1-3.

FIG. 5 schematically illustrates a surgical method for performing a tissue repair.

FIG. 6 schematically illustrates another surgical method for performing a tissue repair.

FIG. 7 illustrates another exemplary locking ferrule.

FIG. 8 illustrates another exemplary locking ferrule.

FIG. 9 schematically illustrates locking a suture relative to the locking ferrule of FIG. 7.

FIG. 10 schematically illustrates yet another surgical method for performing a tissue repair.

FIG. 11 illustrates yet another exemplary locking ferrule.

FIG. 12 schematically illustrates locking a suture relative to the locking ferrule of FIG. 11.

FIG. 13 illustrates an exemplary suture having a varying thickness.

### DETAILED DESCRIPTION

This disclosure relates to systems and methods for performing tensionable knotless tissue repairs. A locking ferrule that includes a one-way locking mechanism may be utilized as part of the tensionable knotless tissue repairs for tensioning and locking one or more strands of suture. These and other features of this disclosure are described in further detail below.

An exemplary locking ferrule for performing tissue repairs may include, inter alia, a body extending between a first end and a second end and including an outer diameter wall, an inner diameter wall, and a cannulation that is circumscribed by the inner diameter wall. An eyelet is connected to the body. A plurality of locking barbs extend into the cannulation and are configured to lock a suture that is received through the eyelet and the cannulation relative to the body.

In any further embodiment, the eyelet is integrally formed with the body.

In any further embodiment, the body is made of a first material, and the eyelet is made of a second material that is different from the first material.

In any further embodiment, the second material includes suture.

In any further embodiment, the eyelet is connected to the second end.

In any further embodiment, the eyelet includes a band that connects to the second end and a suture passage that extends between the band and the second end.

In any further embodiment, the suture is looped around the band.

In any further embodiment, the plurality of locking barbs are angled in a direction toward the eyelet.

In any further embodiment, the plurality of locking barbs are angled in a direction away from the eyelet.

In any further embodiment, the eyelet is connected to the first end.

In any further embodiment, a cap is provided at the second end.

In any further embodiment, the cap includes an increased diameter compared to that of the outer diameter wall of the body.

In any further embodiment, the plurality of locking barbs each protrude inwardly from the inner diameter wall.

In any further embodiment, the plurality of locking barbs are arranged in at least a first row and a second row.

In any further embodiment, a first portion of the plurality of locking barbs of the first row are staggered relative to a second portion of the plurality of locking barbs of the second row.

An exemplary surgical method may include, inter alia, loading a suture through an eyelet of a locking ferrule, passing the suture through a cannulation of the locking ferrule, tensioning the suture in a first direction, and locking the suture within the cannulation to prevent movement of the suture in a second direction.

In any further embodiment, the surgical method includes passing the suture through or around a tissue prior to passing the suture through the cannulation of the locking ferrule.

In any further embodiment, a body of the locking ferrule includes a first end and a second end, and the eyelet is connected to the first end. Passing the suture through the cannulation includes entering the cannulation at the first end and passing the suture through the cannulation in a direction toward the second end of the body.

In any further embodiment, a body of the locking ferrule includes a first end and a second end, and the eyelet is connected to the second end. Passing the suture through the cannulation includes entering the cannulation at the first end and passing the suture through the cannulation in a direction toward the second end of the body.

Another exemplary locking ferrule for performing tissue repairs may include, inter alia, a body including an outer diameter wall, an inner diameter wall, and a cannulation that is circumscribed by the inner diameter wall. A plurality of locking barbs extend into the cannulation and are configured to lock a suture that is received through the cannulation relative to the body. A transverse through-bore extends through the body between a first row and a second row of the plurality of locking barbs.

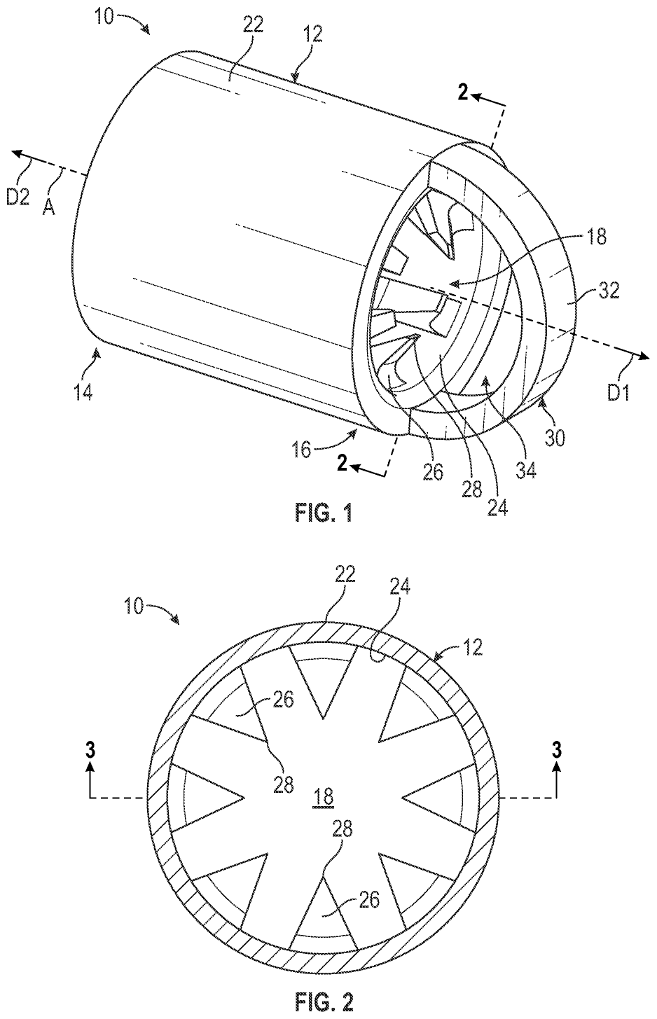

FIGS. 1-4 illustrate an exemplary locking ferrule 10 that can be used when performing various tensionable knotless tissue repairs. For example, the locking ferrule 10 could be utilized during surgical methods for attaching tissue (e.g., ligament, tendon, graft, etc.) to bone, for repairing bone fractures as part of a cerclage technique, for performing tissue reconstruction procedures (e.g., ACL/PCL reconstructions), or for repairing any other type of tissue effect.

The locking ferrule 10 may include a body 12 that extends along a longitudinal axis A between a first end 14 and a second end 16. The body 12 may be tubular shaped and may be constructed from either metallic materials or plastic materials. However, the specific size, shape, and material make-up of the body 12 are not intended to limit this disclosure.

A cannulation 18 may extend through the body 12 and may establish an internal passageway for accommodating one or more strands of suture (see, for example, suture 20 shown in FIG. 4). The cannulation 18 may extend across an entire length of the body 12 and thus extends from the first end 14 to the second end 16. The longitudinal axis A may bisect the cannulation 18.

The body 12 may include an outer diameter wall 22 and an inner diameter wall 24. The outer diameter wall 22 may be smooth or could alternatively include threads, barbs, or other features for facilitating bone fixation. The inner diameter wall 24 may circumscribe the cannulation 18. In some embodiments, the cannulation 18 may taper in a direction toward either the first end 14 or the second end 16.

A plurality of locking barbs 26 may protrude inwardly from the inner diameter wall 24. The locking barbs 26 may therefore occupy at least a portion of the open space of the cannulation 18. In an embodiment, the locking barbs 26 are integrally formed (e.g., molded) features of the body 12 of the locking ferrule 10. The locking barbs 26 may be provided along an entire length of the cannulation 18 or at only select portions thereof. The locking barbs 26 may be either rigid or flexible structures.

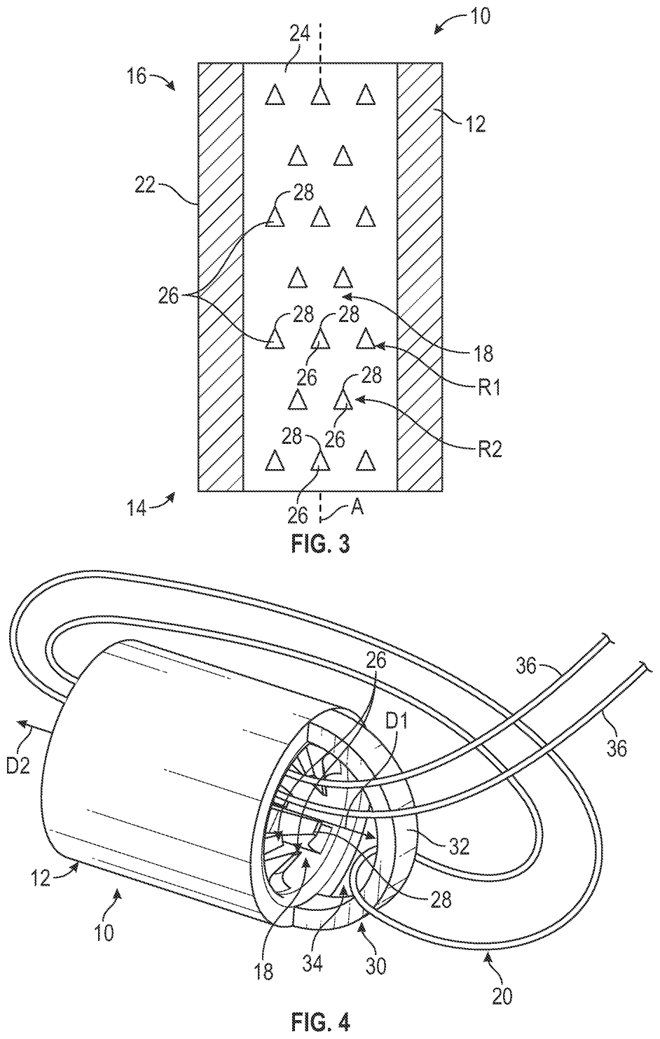

The locking barbs 26 may be arranged in multiple rows along the length of the cannulation 18. For example, the locking barbs 26 may be arranged in a least a first row R1 and a second row R2 (see FIG. 3). In an embodiment, the locking barbs 26 of the second row R2 are staggered relative to the locking barbs 26 of the first row R1 (see FIG. 3).

Each locking barb 26 may include a sharp or pointed tip 28, and, in this implementation, each locking barb 26 may be angled in a direction toward the second end 16. The locking barbs 26 may therefore establish a one-way locking mechanism that permits one or more sutures 20 to pass through the cannulation in a first direction D1 while preventing the suture(s) 20 from being tensioned or otherwise moved in a second direction D2. The staggered relationship of the rows of locking barbs 26 may provide for maximum engagement with the suture 20 once it is passed through the cannulation 18.

The locking ferrule 10 may additionally include an eyelet 30. The eyelet 30 may include a band 32 that is attached or integrally formed with the body 12 of the locking ferrule 10. The band 32 may be made of the same material or a different material (e.g., suture) as the body 12. In an embodiment, the band 32 is connected to the body 12 at or near the second end 16. Therefore, in this implementation, the locking barbs 26 are angled in a direction toward the band 32. An open space extending between the band 32 and the second end 16 of the body 12 may establish a suture passage 34 of the eyelet 30.

Referring now primarily to FIG. 4, with continued reference to FIGS. 1-3, one or more sutures 20 may be loaded through the eyelet 30 and then passed through the cannulation 18. The suture 20 may be FiberWire®, FiberTape®, or any other suitable suture product. FiberWire® and FiberTape® are suture products marketed and sold by Arthrex, Inc. However, other suture products could be utilized for the suture 20 within the scope of this disclosure. The size and type of suture utilized in conjunction with the locking ferrule 10 are not intended to limit this disclosure. The suture 20 could include either a constant thickness or a varying thickness.

The one or more sutures 20 may be accommodated within the suture passage 34 such that the suture(s) 20 is looped around the band 32. The suture 20 may then be inserted into the cannulation 18 at the first end 14 of the body 12 and be passed through the cannulation 18. Once the suture 20 has been passed through the cannulation 18, free ends 36 of the suture 20 may be further tensioned in the first direction D1 to tension and lock the suture 20 in place relative to the locking ferrule 10. The locking barbs 26 prevent the suture 20 from backing up or otherwise moving in the second direction D2. The pointed tips 28 of the locking barbs 26 may interdigitate with the suture 20 to lock the suture 20 and prevent it from moving in the second direction D2.

Although shown as locking a single folded suture 20 in the above implementations, the locking ferrule 10 could be configured to receive and lock multiple strands of suture and/or sutures of varying sizes.

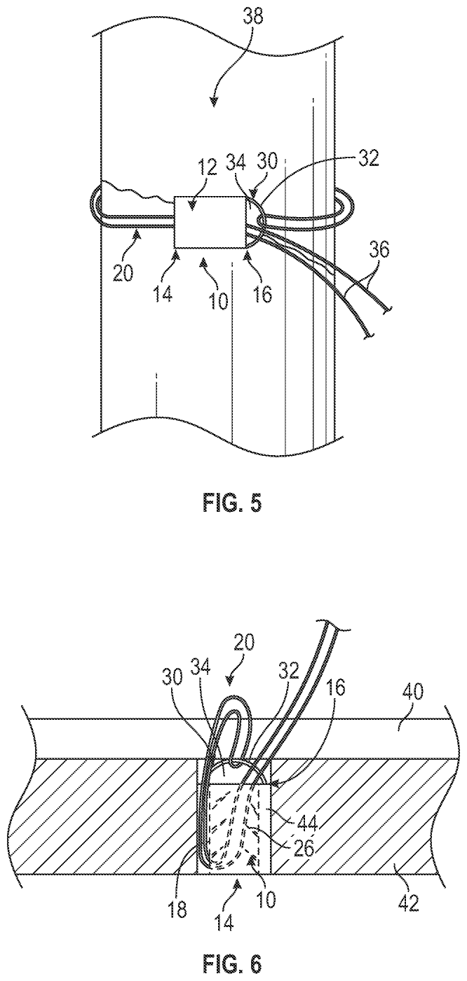

The locking ferrule 10 described above and shown in FIGS. 1-4 may be utilized to tension and knotlessly fixate one or more sutures 20 as part of various surgical methods. FIG. 5 schematically illustrates one such surgical method for repairing a fractured bone 38 as part of a cerclage technique. For example, the suture 20 may be accommodated through the eyelet 30 of the locking ferrule 10 and may then be wrapped around the fractured bone 38. The suture 20 may then be inserted into the first end 14, passed through the cannulation 18 in a direction toward the second end 16, and then tensioned to lock the suture 20 in place, thereby creating a cerclage for allowing the fractured bone 38 to properly heal.

FIG. 6 schematically illustrates another exemplary surgical method that could benefit from the use of the locking ferrule 10. For example, the locking ferrule 10 may be used to attach a tissue 40 to a bone 42, such as when the tissue 40 is torn away from the bone 42. For example, the suture 20 may be accommodated through the eyelet 30 and then be passed through or around the tissue 40 prior to inserting the locking ferrule 10 into an opening 44 formed in the bone 42. The suture 20 may then be passed through the cannulation 18 in a direction that extends from the first end 14 toward the second end 16 and then tensioned to lock the suture 20 in place and thereby approximate the tissue 40 to the bone 42.

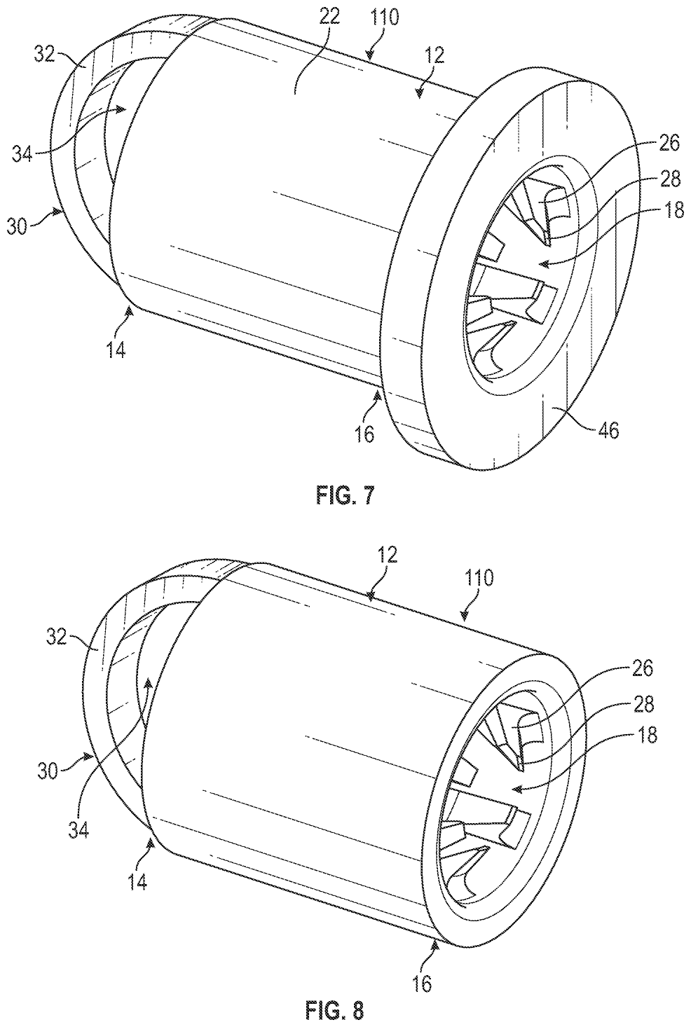

FIG. 7 illustrates another exemplary locking ferrule 110 that can be used when performing various tensionable knotless tissue repairs. The locking ferrule 110 is similar to the locking ferrule 10 discussed above and shown in FIGS. 1-6. However, in this embodiment, the eyelet 30 is provided at or near the first end 14 rather than the second end 16 of the body 12. Therefore, the locking barbs 26 of the cannulation 18 can be angled in a direction away from the band 32 of the eyelet 30.

In an embodiment, a cap 46 is provided at or near the second end 16 of the body 12 of the locking ferrule 10. The cap 46 can therefore be located on an opposite end of the body 12 from the eyelet 30. The cap 46 may include an increased diameter compared to that of the outer diameter wall 22 of the body 12 and is therefore configured to seat relative to a cortex of a bone for providing cortical bone fixation. However, the cap 46 may be omitted from the locking ferrule 110 in some implementations (sec, e.g., FIG. 8).

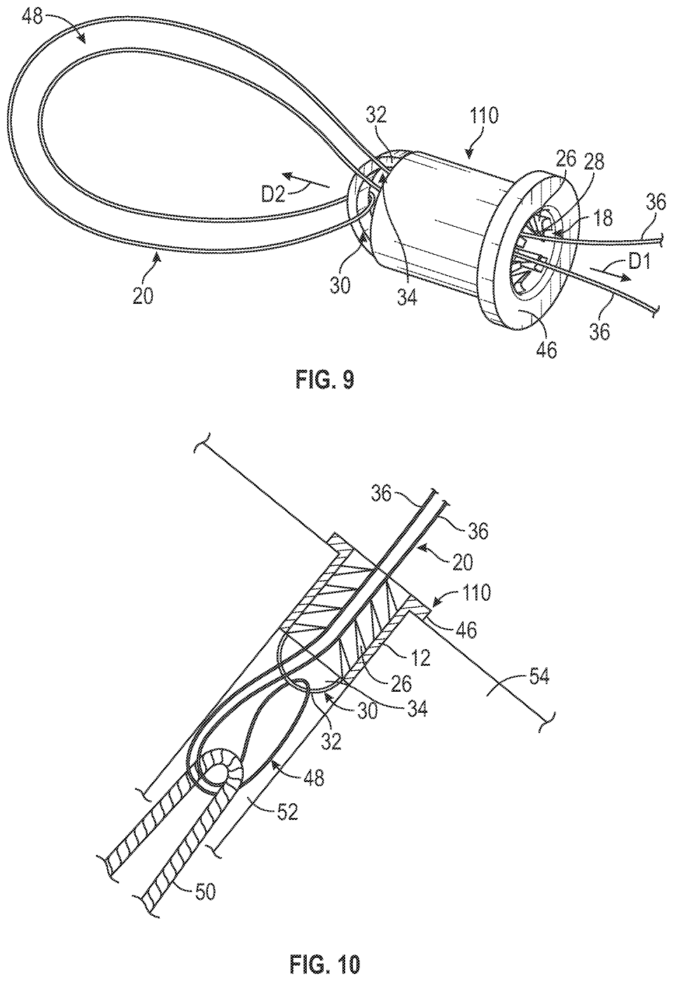

Referring now primarily to FIG. 9, with continued reference to FIG. 7, one or more sutures 20 may be loaded through the eyelet 30 and then passed through the cannulation 18. The suture 20 may first be passed through the suture passage 34 such that the suture(s) 20 is looped around the band 32. The suture 20 may then be inserted into the cannulation 18 through the first end 14 of the body 12 and be passed through the length of the cannulation 18 in a direction toward the second end 16. Once the suture 20 has been passed completely through the cannulation 18, free ends 36 of the suture 20 may be further tensioned in the first direction D1 to tension and lock the suture 20 in place relative to the locking ferrule 110. The locking barbs 26 prevent the suture 20 from backing up or otherwise moving in the second direction D2. The pointed tips 28 of the locking barbs 26 may interdigitate with the suture 20 to lock the suture 20 and prevent it from moving in the second direction D2.

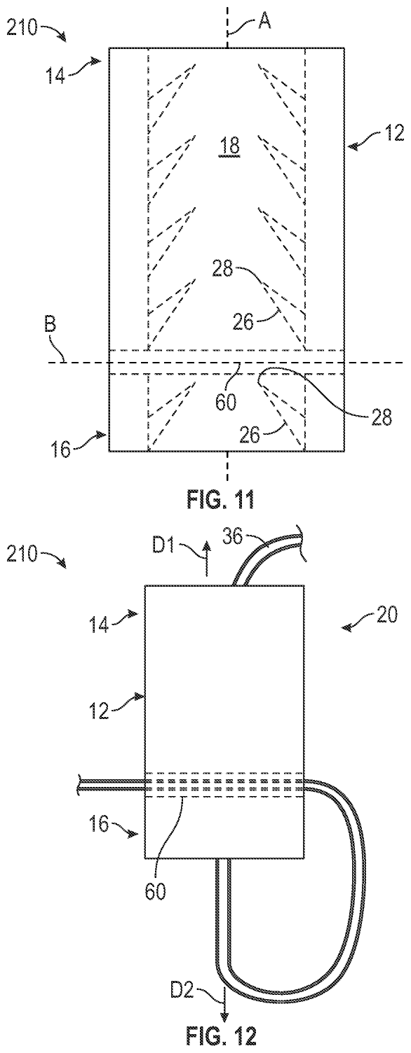

In this embodiment, due at least in part to the positioning of the eyelet 30 at the first end 14, the suture 20 may form a suture loop 48 one it has been looped around the band 32 of the eyelet 30 and passed through the cannulation 18 in a direction that extends from the first end 14 toward the second end 16. A graft 50 may be looped over the suture loop 48 and then be suspended within a bone tunnel 52 of a bone 54 by the locking ferrule 110 (see FIG. 10) as part of a ligament reconstruction procedure. The locking ferrule 110 may be positioned relative to the bone 54 such that the body 12 of the locking ferrule 110 is accommodated within the bone tunnel 52 and the cap 46 is seated relative to the cortex of the bone 54.

FIGS. 11 and 12 illustrates yet another exemplary locking ferrule 210 that can be used when performing various tensionable knotless tissue repairs. In this embodiment, the locking ferrule 210 includes a transverse through-bore 60 rather than the eyelet 30 described above for connecting the suture 20. The transverse through-bore 60 may be formed through the body 12 and may extend along an axis B that is transverse (e.g., perpendicular) to the longitudinal axis A of the body 12. The transverse through-bore 60 may intersect across the cannulation 18 and may extend at a location between adjacent rows of the locking barbs 26. The suture 20 can therefore be passed through the transverse through-bore 60 without engaging any of the locking barbs 26. The transverse through-bore 60 may be positioned closer to either the first end 14 or the second end 16 compared to the opposite end. In the illustrated embodiment, the transverse through-bore 60 is located closer to the second end 16 than the first end 14. However, other configurations are possible within the scope of this disclosure.

The suture 20 may be loaded through the transverse through-bore 60 and then passed through the cannulation 18 from either end depending on the type of procedure being performed. Once the suture 20 has been passed through the cannulation 18, free ends 36 of the suture 20 may be tensioned in the first direction D1 to tension and lock the suture 20 in place relative to the locking ferrule 210. The locking barbs 26 prevent the suture 20 from backing up or otherwise moving in the second direction D2.

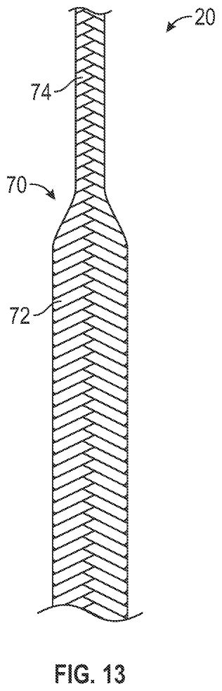

FIG. 13 illustrates an exemplary suture 20 that may be utilized in any of the implementations discussed above. The suture 20 may include a varying thickness. The suture 20 may therefore include one or more tapered regions 70 where the suture 20 transitions between a thickened section 72 and a thinned section 74. The thinned sections 74 can facilitate passing the suture 20 through the cannulation 18 and/or the through-bore 60, such as via a suitable suture loader that can be provided as part of a suture locking system that includes the locking ferrule 10, for example, and the thickened sections 72 can provide greater surface area for the locking barbs 26 to engage in order to sufficiently lock the suture 20 relative to the locking ferrule 10.

In an embodiment, the thickened sections 72 of the suture 20 are about twice as thick as the thinned sections 74. However, other ratios (e.g., 1.5:1, 3:1, etc.) between the relative thicknesses (e.g., outer diameters) of the thickened section 72 and the thinned section 74 are contemplated within the scope of this disclosure.

The locking ferrules of this disclosure may be utilized for performing various tensionable knotless tissue repairs. The locking ferrules provide for tensioning and retensioning suture(s) at various points of the tissue repair, including subsequent to implantation of the accompanying fixation devices, thus providing numerous advantages over prior tissue repair techniques.

Although the different non-limiting embodiments are illustrated as having specific components or steps, the embodiments of this disclosure are not limited to those particular combinations. It is possible to use some of the components or features from any of the non-limiting embodiments in combination with features or components from any of the other non-limiting embodiments.

It should be understood that like reference numerals identify corresponding or similar elements throughout the several drawings. It should further be understood that although a particular component arrangement is disclosed and illustrated in these exemplary embodiments, other arrangements could also benefit from the teachings of this disclosure.

The foregoing description shall be interpreted as illustrative and not in any limiting sense. A worker of ordinary skill in the art would understand that certain modifications could come within the scope of this disclosure. For these reasons, the following claims should be studied to determine the true scope and content of this disclosure.

What is claimed is:

1. A locking ferrule for performing tissue repairs, comprising:
a body extending between a first end and a second end and including an outer diameter wall, an inner diameter wall, and a cannulation that is circumscribed by the inner diameter wall;
an eyelet connected to the body near the first end or the second end; and
a plurality of locking barbs extending into the cannulation and configured to lock a suture that is received through the cannulation relative to the body.

2. The locking ferrule as recited in claim 1, wherein the eyelet is integrally formed with the body.

3. The locking ferrule as recited in claim 1, wherein the body is comprised of a first material, and the eyelet is comprised of a second material that is different from the first material.

4. The locking ferrule as recited in claim 3, wherein the second material includes suture.

5. The locking ferrule as recited in claim 1, wherein the eyelet is connected to the second end.

6. The locking ferrule as recited in claim 5, wherein the eyelet includes a band that connects to the second end and a suture passage that extends between the band and the second end.

7. The locking ferrule as recited in claim 6, wherein the suture is looped around the band.

8. The locking ferrule as recited in claim 1, wherein the plurality of locking barbs are angled in a direction toward the eyelet.

9. The locking ferrule as recited in claim 1, wherein the plurality of locking barbs are angled in a direction away from the eyelet.

10. The locking ferrule as recited in claim 9, wherein the eyelet is connected to the first end.

11. The locking ferrule as recited in claim 10, comprising a cap provided at the second end.

12. The locking ferrule as recited in claim 11, wherein the cap includes an increased diameter compared to that of the outer diameter wall of the body.

13. The locking ferrule as recited in claim 1, wherein the plurality of locking barbs each protrude inwardly from the inner diameter wall.

14. The locking ferrule as recited in claim 13, wherein the plurality of locking barbs are arranged in at least a first row and a second row.

15. The locking ferrule as recited in claim 14, wherein a first portion of the plurality of locking barbs of the first row are staggered relative to a second portion of the plurality of locking barbs of the second row.

16. A surgical method, comprising:
loading a suture through an eyelet of a locking ferrule;
passing the suture through a cannulation of the locking ferrule;
tensioning the suture in a first direction; and
locking the suture within the cannulation to prevent movement of the suture in a second direction.

17. The surgical method as recited in claim 16, comprising:
passing the suture through or around a tissue prior to passing the suture through the cannulation of the locking ferrule.

18. The surgical method as recited in claim 16, wherein a body of the locking ferrule includes a first end and a second end, and the eyelet is connected to the first end, and further wherein passing the suture through the cannulation includes:
entering the cannulation at the first end and passing the suture through the cannulation in a direction toward the second end of the body.

19. The surgical method as recited in claim 16, wherein a body of the locking ferrule includes a first end and a second end, and the eyelet is connected to the second end, and further wherein passing the suture through the cannulation includes:
entering the cannulation at the first end and passing the suture through the cannulation in a direction toward the second end of the body.

20. A locking ferrule for performing tissue repairs, comprising:
a body including an outer diameter wall, an inner diameter wall, and a cannulation that is circumscribed by the inner diameter wall;
a plurality of locking barbs extending into the cannulation and configured to lock a suture that is received through the cannulation relative to the body; and
a transverse through-bore that extends through the body between a first row and a second row of the plurality of locking barbs.
